(19) United States
(12) Patent Application Publication
(10) Pub. No.: US 2020/0348231 A1
(43) Pub. Date: Nov. 5, 2020

(54) MOLECULARLY IMPRINTED FLUORESCENCE SENSOR BASED ON CARBON DOTS FOR DETECTING CHLORAMPHENICOL AND ITS PREPARATION METHOD AND ITS APPLICATION

(71) Applicant: Jiangnan University, Wuxi (CN)

(72) Inventors: Hang YU, Wuxi (CN); Shishi CHEN, Wuxi (CN); Yunfei XIE, Wuxi (CN); Yahui GUO, Wuxi (CN); Yuliang CHENG, Wuxi (CN); Weirong YAO, Wuxi (CN)

(21) Appl. No.: 16/460,581

(22) Filed: Jul. 2, 2019

(30) Foreign Application Priority Data
May 5, 2019 (CN) 201910368009.0

(51) Int. Cl. G01N 21/64; B01L 3/00; B01J 20/26; B01J 20/30; G01N 33/02

(52) U.S. Cl. CPC G01N 21/6428; B01L 3/52; B01J 20/268; B01L 2200/18; G01N 33/02; B01L 2300/123; B01L 2200/12; B01J 20/3085

(57) ABSTRACT

The present invention discloses a molecularly imprinted fluorescence sensor based on carbon dots for detecting chloramphenicol (CAP) and its preparation method and its application. This invention uses carbon dots (CD) as fluorescence carrier and molecularly imprinted membrane (MIP) as enrichment container to synthesize fluorescent molecularly imprinted material with core-shell structure so that to achieve a rapid and specific detection of CAP. The reverse micro-emulsion method was used, firstly a reverse microemulsion system was established, CAP was used as a template molecule in the water phase, and a molecularly imprinted membrane was synthesized on the surface by using CD as a carrier. The prepared material is nano-sized microspheres with excellent water-dispersibility and stability. It has good sensitivity to the target substance CAP, rapid detection speed, strong specific selectivity, strong chemical stability and low cost, and has good application prospects in the detection of CAP.

### TECHNICAL FIELD

The present invention relates to a novel fluorescence sensor comprising carbon dots as fluorescence probes and molecularly imprinted membrane as enrichment container for detecting chloramphenicol and its preparation method and application, and belongs to the field of food analytical methods.

### BACKGROUND

Chloramphenicol (CAP) has been widely used worldwide as a broad-spectrum antibiotic with a relatively strong antibacterial ability. However, CAP has become the first antibiotic that was banned in edible animals due to its serious toxic effects, e.g. toxicity on inhibiting bone marrow hematopoietic function, genotoxicity, and gray baby syndrome etc. Quantification of CAP is very challenging, especially when an extremely low concentration of CAP (1-10 μg/kg) is contained in various samples with complex matrices, such as the food systems. Therefore, CAP monitoring requires novel, rapid and accurate cleaning and enrichment methods.

Conventional detection methods of CAP include microbiological method, chromatographic analysis, spectrophotometric analysis, enzyme-linked immuno-sorbent assay, etc. The chromatographic analysis is one of the most popular methods for detecting CAP; however, due to a high price of devices, and complexity of pre-treatment of samples, it would cause a huge waste of organic solvents and increase the cost of detection. Some other disadvantages of conventional detection methods, such as relatively low sensitivity, false-positive results, time consuming, etc., may happen simultaneously.

Due to limitations of conventional analysis, it is a trend to develop some portable devices for detecting CAP rapidly and correctly. Up to now, some types of molecularly imprinted membrane (MIP) have been synthesized and applied to pre-treatment of samples. It is considered as an innovative enrichment method combined with spectrophotometric analysis that achieves a rapid and accurate detection of CAP in food samples. Quantum dots (QD) are new types of zero-dimensional nanomaterials with many advantages and great potentials in the field of biochemical sensing, e.g. fluorescence adjustment, wide excitation and narrow emission, anti-light bleaching, high detection sensitivity, etc. Metal quantum dots (MQD) are the most commonly used because of excellent optical properties, such as CdSe, CdTe, etc. The combination of MQD-MIP and fluorescence sensor has been successfully utilized to detect CAP. It is reported that the sensitivity of MQD-MIP with fluorescence detection is relatively high; however, the reaction time is relatively long (15 min). Moreover, toxic heavy metals would cause a serious environmental pollution when leakage of heavy metal ions is happened.

### SUMMARY

In order to solve the problems described above, the present invention developed a novel fluorescence sensor comprising carbon dots (CDs) as fluorescence probes and MIP as enrichment containers (i.e. MIP@CD) for detecting CAP. Compared with MQD, the CD was environment-friendly without any toxicity, therefore, it can be a desired replacement of MQD. The prepared fluorescence sensor was then synthesized by reversed micro-emulsion, which had an excellent dispersibility and stability in water. The fluorescence sensor also has a reasonably high sensitivity to the target substance, such as CAP. Therefore, the fluorescence sensor of present invention is suitable for rapid detection of CAP in complex matrix, e.g. food systems.

The embodiment of the invention provides a method for the rapid detection of CAP through a newly developed MIP@CD fluorescence sensor. The method comprises the following steps:

(1) One-step hydrothermal synthesis of nitrogen-doped CD: completely dissolving citric acid and urea into ultrapure water through sonication; transferring the mixture into a well-sealed reactor; heating the reactor and cooling down to the room temperature; adding acetone into the reactor, centrifuging and subsequently undergoing vacuum drying for obtaining the CD. In certain specific embodiments, the molar-based ratio of citric acid and urea is 1:3. The temperature of the heating process is 160° C. and the duration 4 h.
(2) Establishment of reversed micro-emulsion system: mixing oil phase cyclohexane, cosurfactant hexanol, and surfactant triton X-100, and stirring the mixture on a magnetic stirrer for 20 min; adding 1 mg/mL of CD solution and stirring for further 20 min. In certain specific embodiments, the volumetric ratio of cyclohexane, hexanol, triton X-100, and CD solution is 7.50:1.80:1.77:1.00. The Magnetic stirring speed is 800 r/min.
(3) Mixing 3-aminopropyltriethoxysilane and CAP, and pre-polymerizing the mixture at 4° C. for 2 h. The molar-based ratio of 3-aminopropyltriethoxysilane and CAP is 5:1 and pre-polymerized solution is obtained.
(4) Adding tetraethyl silicate and ammonia as a catalyst into the reversed micro-emulsion system in the step (2), and stirring for 2 h; mixing it with the pre-polymerized solution obtained in the step (3), and stirring for 24 h; for each mL of CD solution, 0.20 mmol of 3-aminopropyltriethoxysilane, 0.04 mmol of CAP, 0.80 mmol of tetraethyl silicate, and 60 μL of ammonia are need to be added. Finally adding acetone into the reactor, centrifuging and subsequently undergoing vacuum drying for obtaining MIP@CD precursor.
(5) Ultrasound-extracting of template molecular CAP in the MIP@CD precursor with a methanol-acetic acid solvent, and keeping the ultrasonic extraction until the template molecular CAP becomes undetectable at 274 nm under the UV-Vis.
(6) Vacuum drying of MIP@CD at 60° C.; and obtaining the MIP@CD fluorescence sensor.

In certain specific embodiments, the particle size of CD is 2-5 nm; and the particle size of the MIP@CD fluorescence sensor is 5-15 nm.

Application of the MIP@CD fluorescence sensor to CAP detection:

(A) Setting parameters of fluorescence detection as follows: excitation wavelength of 330 nm, excitation and emission slit width of 10 nm.
(B) Preparing 100 μg/mL water-dispersion of fluorescence sensor; mixing the pretreated CAP sample with the 100 μg/mL water-dispersion of fluorescence sensor at a volume ratio of 1:1. The fluorescence detection is conducted after 5 min.

The MIP@CD fluorescence sensor can be used for separation, enrichment and fluorescence quantitative detection of CAP in complex food matrices.

Compared with the prior art, the present invention has following advantageous effects:

1) The present invention combines and takes full advantage of the unique properties of MIP and CD. Compared with using MIP alone, the present invention has developed the fluorescence detecting probe to achieve the detection of target compound. Compared with using CD alone, the present invention has developed an excellent system to enrich the target compound, which can achieve the quantitative detection of target at a low concentration. Compared with MQD-MIP materials, the use of CD instead of MQD is more environmentally friendly.
2) The MIP@CD fluorescence sensor prepared in present in invention has an excellent water-dispersibility and stability, and also has good sensitivity and high selectivity to the target substance such as CAP. MIP@CD fluorescence sensor of present invention can achieve to a fast and sensitive detection on trace of CAP in the food system.
3) The sensor material has a high mechanical strength, and strong chemical stability; it is also reusable with a relative low cost and that makes it as a potential detecting sensor in the detection of food system.

### BRIEF DESCRIPTION OF THE DRAWINGS

FIG. 1 shows Transmission Electron Microscope (TEM) images of CD (A and B), and MIP@CD fluorescence sensor (C and D).

FIG. 2 shows fluorescence emission spectra of CD (A) and MIP@CD (B) excited at excitation wavelengths ranging from 300 to 350 nm at 10 nm interval.

FIG. 3 shows selectivity of MIP@CD and non-imprinted polymers-coated CD (NIP@CD) to CAP, thiamphenicol (TAP), florfenicol (FF), sodium succinate chloramphenicol (HS-CAP), sulfadiazine (SD), neomycin sulfate (NED) and amoxicillin (AMO).

### DETAILED DESCRIPTION

As mentioned above, for overcoming the defects of the prior art, the inventor puts forwards the technical solution of the invention through lone-term study and mass practices, and a fluorescence sensor of MIP@CD for achieving a rapid and accurate determination of CAP is mainly provided. Generally, the method comprises the first step of synthesizing nitrogen-doped CD through the one-step hydrothermal reaction, the second step of synthesizing of MIP fluorescence sensor based on CD, and the third step of characterizing and evaluating performance of MIP@CD on detecting CAP. According to the method of the invention, the detection speed is high (within 5 min), the raw material is environment-friendly (without heavy metals), the detection cost is low, and the quantification of CAP in the complex food matrix (e.g. Carassius auratus as a fish sample) can be determined rapidly and accurately.

The detailed description of the present invention is as follows:

### Embodiment 1 Preparation of the MIP@CD Fluorescence Sensor

In certain specific embodiments, the method for synthesizing MIP@CD fluorescence sensor comprises the following steps:

### 1. Preparation of Nitrogen Doped CD:

4 mmol citric acid (as carbon source) and 12 mmol urea (as nitrogen source; molar ratio: 1:3) were completely dissolved into ultrapure in 20 mL water through sonication; the obtained mixture was transferred to a well-sealed 50 mL reactor; the reactor was heated to 160° C. and maintained for 4 h, and then cooled down to the room temperature; acetone was added into the reactor for demulsification, and centrifuged at 5000 r/min for 5 min, and then the precipitate obtained by centrifugation was underwent vacuum drying for obtaining the CD.

### 2. Synthesizing MIP@CD Fluorescence Sensor:

7.50 mL cyclohexane (in oil-phase), 1.80 mL hexanol (cosurfactant), and 1.77 mL triton X-100 (surfactant; volume ratio: 7.50:1.80:1.77) were mixed in a 100 mL stopper flask, and then the mixture was stirred in a magnetic stirrer for 20 min with the stirring speed of 800 r/min. 1 mL of 1 mg/mL CD solution was added and stirred for further 20 min. 0.80 mmol tetraethyl silicate and 60 μL ammonia were added into the mixture, and stirred for further 2 h. A pre-polymerized solution comprising 0.20 mmol 3-aminopropyltriethoxysilane and 0.04 mmol CAP (molar ratio: 5:1), which has been placed at 4° C. for 2 h, was added and stirred for 24 h before 10 mL acetone was added into the reactor, the obtained mixture was centrifuged and the precipitate obtained by centrifugation was collected. Subsequently undergoing vacuum drying at 60° C. for obtaining MIP@CD precursor. The MIP@CD precursor was underwent an ultrasound-extracting of template molecular CAP with a methanol-acetic acid solvent until the template molecular CAP was undetectable in the MIP@CD at 274 nm under the UV-Vis; and lastly MIP@CD was vacuum dried at 60° C. for obtaining the MIP@CD fluorescence sensor.

### 3. Synthesizing NIP@CD Fluorescence Sensor as a Control:

The NIP@CD fluorescence sensor as a control was prepared according to the step (2) above-described without adding CAP in the synthesizing of NIP@CD.

### Embodiment 2 The Characteristics and Evaluation of the MIP@CD Fluorescence Sensor

In certain specific embodiments, the synthesized fluorescence sensor MIP@CD is characterized and evaluated its functions as follows:

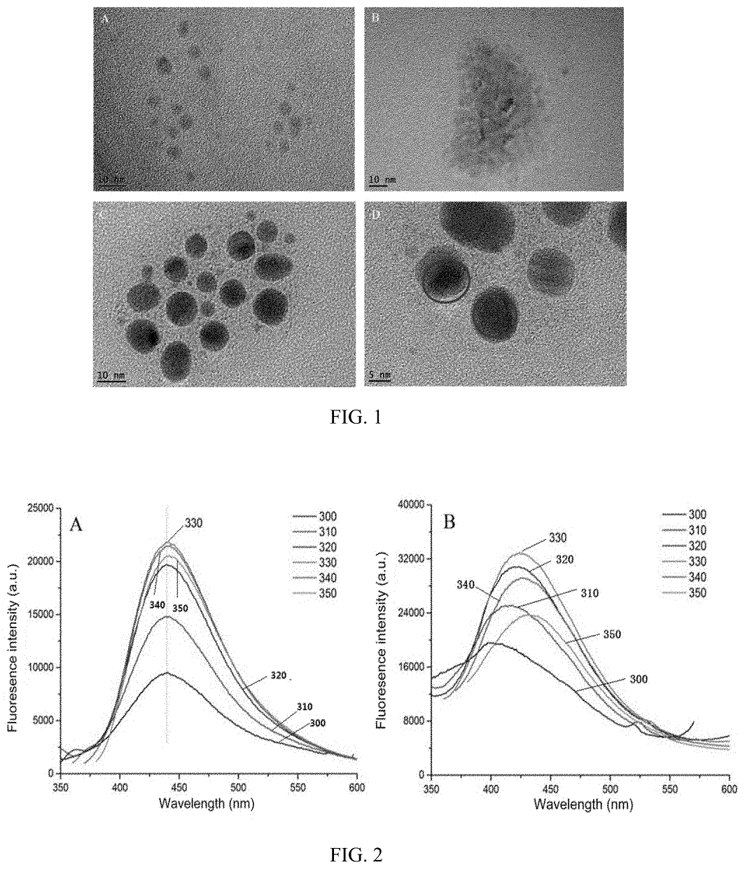

As shown in FIG. 1, A and B are Transmission Electron Microscope (TEM) images of carbon dot (CD); C and D are TEM images of MIP@CD fluorescence sensor. The TEM image (FIG. 1A) shows the CD has a particle size 2-5 nm approximately with lattice structure. The MIP@CD fluorescence sensor in FIG. 1C has a bigger particle size ranging from 5-15 nm, which is mainly due to the extra MIP membrane wrapping outside the CD. In FIG. 1D, a deep color in MIP@CD microsphere refers to the CD wrapped in middle, which again proved that the MIP@CD structure is well established. Compared with the aggregation of CD (FIG. 1B), the MIP@CD (FIG. 1C) showed a better dispersibility. This phenomenon can be attributed to the strong van der Waals force between the surface groups of CD which tends to cause aggregation; and the presence of MIP membrane can block such force between the CD, so that prevents the aggregation of CDs.

As shown in FIG. 2, the fluorescence emission intensity of CD and MIP@CD is first increased and then decreased when the excitation wavelength is increased from 300 to 350 nm and reaches the strongest fluorescence emission when the excitation wavelength is 330 nm. It is noticeable that the emission wavelength of CD remains constant at 440 nm; therefore, CD does not show the excitation wavelength dependence. On the contrary, the emission of MIP@CD sensor material exhibits a red-shift phenomenon with the increase of excitation wavelength which means that MIP@CD has excitation wavelength dependence. It is proved that optical properties of MIP@CD sensor come from CD; however, differences on optical properties of MIP@CD are mainly attributed to the combination of MIP.

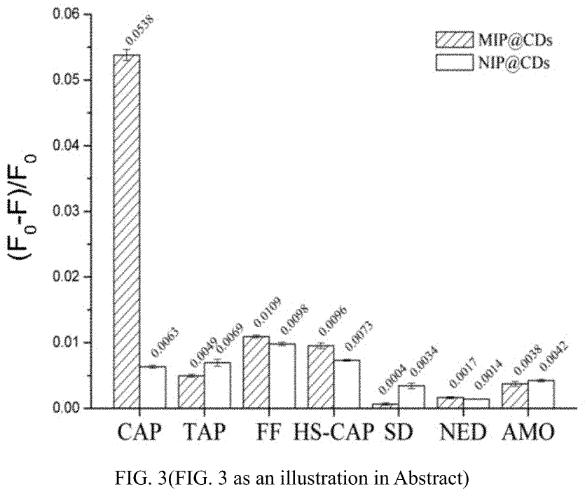

As shown in FIG. 3, MIP@CD shows a good selectivity of CAP. It can be observed that the CAP quenching rate of MIP@CD sensor is about 4 times higher than that of other antibiotics and the quenching effect of antibiotics on MIP@CD is much stronger than that on NIP@CDs.

### Embodiment 3 The Application of the MIP@CD Fluorescence Sensor in the Detection of CAP

Measurement of CAP through MIP@CD fluorescence sensor is conducted with excitation wavelength of 330 nm and emission slit width of 10 nm. The MIP@CD fluorescence sensor is dissolved into deionized water and prepared a water-dispersion of MIP@CD fluorescence sensor at 100 μg/mL Mixing the dispersion with standard CAP solution or CAP sample solution at a volume ratio of 1:1. The fluorescence detection is conducted after standing the mixture for 5 min.

Results shows that the CAP quenching effect of the MIP@CD fluorescence sensor follows Stern-Volmer equation, i.e. F₀/F=Kₛᵥ·[Q]+1, wherein F₀ is the fluorescence intensity without adding CAP; F is the fluorescence intensity with addition of CAP; F₀/F is the relative fluorescence intensity; [Q]is the concentration of CAP; Kₛᵥ is the fluorescence quenching constant. It can be seen that the fluorescence intensity gradually decreases as the concentration of CAP increases.

When the concentration of CAP is ranging from 0 to 1.25×10²μmol/L, there are two linear relations between concentration of CAP and the relative fluorescence intensity F₀/F. The linear regression equation of F₀/F=5.510C+1.021 (R²=0.9969) is suitable for a relatively low concentration of CAP ranging from 1.50×10⁻³ to 1.50×10⁻² μmol/L; when a high concentration of CAP ranging from 1.50×10 ⁻²to 1.25×10²μmol/L, the linear regression equation F₀/F=0.002C+1.118 (R²=0.9661) shows a better fitting performance. This phenomenon may be attributed to the uneven size of MIP@CD material and imprinted cavity size. Usually, due to a low concentration of CAP in food samples, the linear regression equation of low concentration is more suitable for practical application. The detection limit is determined based on the 3σ/Kₛᵥ(n=9) standard wherein Kₛᵥ is the slope of the linear calibration, σ is the standard deviation of the blank signal. Based on the linear equation of low concentration, the detection limit is determined to be 12.83 nmol/L. The relative standard deviation (RSD) of the blank sample is 2.55% after 9 repeated tests.

The CAP in Carassius auratus is detected by the sensor in order to further study the practical application of the MIP@CD fluorescence sensor. Recovery studies are carried out by incorporating samples with CAP ranging from 0.5-5.0 μg/L. Results are shown in Table 1.

TABLE 1
Quantification of CAP in Carassius auratus (n = 3).

| Concentration of CAP (μg/L) | | | |
|---|---|---|---|
| Addition value | Detected value | Recovery rate (%) | RSD (%) |
| 0 | Not detected | — | — |
| 0.5 | 0.48 | 95.16 | 1.64 |
| 2.5 | 2.56 | 102.53 | 5.63 |
| 5.0 | 4.50 | 90.02 | 1.03 |

Recovery rates are ranging from 90.02%-102.53%, and RSD are less than 5.70% in Carassius auratus. Therefore, the MIP@CD fluorescence sensor would become an effective method for determining CAP at the low concentration that makes it more partible in real scenarios.

Although the present invention has been disclosed in the above preferred embodiments, the present invention is not limited thereto, and various modifications and changes can be made thereto without departing from the spirit and scope of the invention by those skilled in the field. Therefore, the protection scope of the invention should be determined by the scope of the claims.

1. A method for preparing MIP@CD fluorescence sensor for detecting CAP, comprising the steps:
(1) one-step hydrothermal synthesis of nitrogen-doped CD: taking citric acid as carbon source and urea as nitrogen source, completely dissolving citric acid and urea into ultrapure water through sonication; transferring the mixture into a well-sealed reactor; heating the reactor and cooling down to the room temperature; adding acetone into the reactor for demulsification, centrifuging and vacuum drying the precipitate obtained by centrifugation for obtaining the CD; the molar ratio of citric acid and urea is 1:3;
(2) establishment of reversed micro-emulsion system: mixing oil phase cyclohexane, cosurfactant hexanol, and surfactant triton X-100, and stirring the mixture on a magnetic stirrer for 20 min; adding 1 mg/mL of CD solution and stirring for further 20 min; the volume ratio of cyclohexane, hexanol, triton X-100, and CD solution is 7.50:1.80:1.77:1.00;
(3) mixing 3-aminopropyltriethoxysilane and CAP, and pre-polymerizing them at 4° C. for 2 h; the molar ratio of 3-aminopropyltriethoxysilane and CAP is 5:1;
(4) adding tetraethyl silicate as a cross-linking agent and ammonia as a catalyst into the reversed micro-emulsion system in the step (2) and stirring for 2 h; mixing the mixture and the pre-polymerized solution in the step (3) and stirring for 24 h; for each milliliter of CD solution, 0.20 mmol of 3-aminopropyltriethoxysilane, 0.04 mmol of CAP, 0.80 mmol of tetraethyl silicate, and 60 μL it of ammonia are added; adding acetone, into the reactor, centrifuging and collecting the precipitate of MIP@CD precursor;
(5) ultrasound-extracting of template molecular CAP in the MIP@CD precursor with a methanol-acetic acid solvent until the template molecular CAP is undetectable at 274 nm under the UV-Vis;
(6) vacuum drying MIP@CD at 60° C. and obtaining the MIP@CD fluorescence sensor.

2. The method according to claim 1, wherein the particle size of CD is 2-5 nm.

3. The method according to claim 1, wherein the particle size of MIP@CD fluorescence sensor is 5-15 nm.

4. The method according to claim 1, wherein the temperature of the heating process in step (1) is 160° C., and the duration is 4 h.

5. The method according to claim 3, wherein the temperature of the heating process in step (1) is 160° C., and the duration is 4 h.

6. The method according to claim 1, wherein the stirring speed of the magnetic stirrer in step (2) is 800 r/min.

7. The method according to claim 4, characterized in that wherein the stirring speed of the magnetic stirrer in step (2) is 800 r/min.

8. A MIP@CD fluorescence sensor for detecting CAP, wherein the MIP@CD fluorescence sensor combines MIP having specific recognition and enrichment function with fluorescent sensitive, environmentally friendly and non-toxic CD; the CD is a nitrogen-doped CD; the fluorescence sensor is prepared by synthesizing MIP on the surface of CD through the reversed micro-emulsion and eluting the template molecular CAP.

9. An application of the MIP@CD fluorescence sensor obtained by the method according to claim 1 in detecting CAP, wherein,
(A) setting excitation wavelength of 330 nm, excitation and emission slit width of 10 nm;
(B) preparing 100 μg/mL water-dispersion of fluorescence sensor; mixing the pretreated CAP sample with the 100 μg/mL water-dispersion of fluorescence sensor at a volume ratio of 1:1;
conducting the fluorescence detection after 5 min.

10. The application according to claim 9, wherein the MIP@CD fluorescence sensor is used in the separation, enrichment, and fluorescence quantitative detection of CAP in complex food matrices.
